(19) United States
(12) Patent Application Publication
(10) Pub. No.: US 2026/0102582 A1
(43) Pub. Date: Apr. 16, 2026

(54) SYSTEM FOR REDUCING HYPERSENSITIVITY AND METHOD OF CONTROLLING A DEVICE THAT OUTPUTS A STIMULUS TO A SUBJECT

(71) Applicant: ITS-EASY BOERLIN, Reinach (CH)

(72) Inventor: Elena BOERLIN, Reinach (CH)

(21) Appl. No.: 19/120,633

(22) PCT Filed: Oct. 12, 2023
(86) PCT No.: PCT/EP2023/078300

§ 371 Date: Apr. 11, 2025

(30) Foreign Application Priority Data
Oct. 14, 2022 (CH) CH001218/2022

(51) Int. Cl. A61M 21/00

(52) U.S. Cl. CPC A61M 21/00; A61M 2021/0027; A61M 2021/005

(57) ABSTRACT

A system for reducing hypersensitivity comprises a storage device configured to store a plurality of stimuli and a control circuit coupled to the storage device and configured to select a stimulus of the plurality of stimuli and cause the selected stimulus to be output to a wearer of virtual reality goggles via a visual output device and/or an aural output device of the VR goggles. The storage device is configured to store difficulty levels for the plurality of stimuli. The control circuit is configured to use the stored difficulty levels to select the stimulus.

### TECHNICAL FIELD

The present invention relates to a system according to the preamble of independent claim 1 and more particularly to a system that can be used for reducing hypersensitivity. The invention also relates to a method of controlling virtual reality (VR) goggles.

Such a system can be used for treating hypersensitivity of a subject with autism spectrum disorder (ASD), attention deficit hyperactivity disorder (ADHD), traumatic brain injury (TBI), stroke, fibromyalgia, post-traumatic stress disorder (PTSD), complex post-traumatic stress disorder (cPTSD), developmental trauma disorder (DTD), other mental disorders. The system is configured in such a way that it can be operated in a residential setting, obviating the need for the treatment to be performed in a dedicated medical setting such as a hospital or doctor's office.

The control method is useful in operating the system under the control of a control circuit that may be provided in a computer or game console.

### BACKGROUND ART

KR 102078792 B1 discloses an apparatus for treating Asperger. Y. Cheng et al., “Using a 3D Immersive Virtual Environment System to Enhance Social Understanding and Social Skills for Children With Autism Spectrum Disorders”, Focus on Autism and Other Development Disabilities, Vol. 30, Issue 4, http://journals.sagepub.com/doi/pdf/10.1177/1088357615583473#examines the effect of virtual reality immersion on improving social behavior. Both documents relate to social behavior patterns and do not allow sensory filters to be trained in a systematic manner to reduce hypersensitivity.

US 2019/0008441 A1 discloses a diagnostic system and method.

U.S. Pat. No. 6,425,764 B1, which forms the basis for the preamble of claim 1, is yet another example of a system that uses virtual reality immersion therapy for treatment purposes. The system is not configured to improve sensory filters in a systematic manner.

L. Pavanatto et al., “Get the job! An immersive simulation of sensory overload”, 2020 IEEE Conference on Virtual Reality and 3D User Interfaces Abstracts and Workshops (VRW), pp. 509-510 discloses techniques that allow a sensory overload experience to be simulated.

Mayer L. Max and John C. Burke, “Virtual Reality for Autism Communication and Education, with Lessons for Medical Training Simulators”, Studies in Health Technology and Informatics, Vol. 39, pp. 46-53 discloses adapting Virtual Reality (VR) technologies to teach children with autism new coping skills that may then be generalized in their everyday lives.

U.S. Pat. No. 10,809,796 B2 discloses a technique of providing VR stimuli. U.S. Pat. No. 10,809,796 B2 does not provide a mechanism that allows sensory filters to be trained in a systematic way. Even in consideration of this document there is still a need for enhanced techniques that allow a control circuit to select stimuli for presentation in such a manner that sensory filters are gradually trained.

US 2016/275805 A1, US 2020/356136 A1, and US 2020/234827 A1 disclose further examples of techniques in which output is provided to a subject. The output may be adjusted. Even in consideration of these documents there is still a need for enhanced techniques that allow a control circuit to select stimuli for presentation in such a manner that sensory filters are gradually trained.

CN 110 891 638 A discloses a virtual reality (VR) experience generating system which includes a medical procedure library storing sequences of operational actions of one or more medical procedures. This VR experience generating system does not provide a mechanism that allows a control circuit to select stimuli for presentation in such a manner that sensory filters are trained.

The techniques discussed in the above publications aim at improving social skills, emulating sensory overload situations, or provide diagnostic tools. These techniques do not allow sensory filters to be trained in a systematic manner to reduce hypersensitivity. The conventional techniques also, at least in part, require dedicated medical settings, such as hospitals, which make it challenging to integrate these techniques in everyday life settings in which a person suffering from hypersensitivity feels more relaxed and more at ease. The conventional techniques also, at least in part, require the presence of a medical professional, which makes them complex to apply.

Therefore, there is a need for a system for treating hypersensitivity, which allows sensory filters to be trained. There is in particular a need for a system that is configured to operate in a residential setting and without the on-site presence of a medical professional.

### Disclosure of the Invention

According to the invention this need is settled by a system as it is defined by the features of independent claim 1, by a control method as it is defined by the features of independent claim 15, and by a machine-readable instruction code as it is defined by the features of independent claim 17. Preferred embodiments are recited in the dependent claims.

In one aspect, the invention is a system for reducing hypersensitivity of a subject. The system comprises a storage device configured to store a plurality of stimuli and a control circuit coupled to the storage device and configured to select a stimulus of the plurality of stimuli and cause the selected stimulus to be output to the subject. The storage device is configured to store difficulty levels for the plurality of stimuli. The control circuit is configured to use the stored difficulty levels to select the stimulus.

The system is configured to output a stimulus selected in accordance with assigned difficulty levels, thereby providing an objective criterion for identifying a suitable stimulus. The system uses a suitable output modality for the stimulus, and the control circuit and storage device may be provided in a personal computer (PC) or game console. For visual stimulation, the output modality may be or may comprise display(s) or other optical output device(s) of virtual reality (VR) goggles. For aural stimulation, the output modality may be or may comprise speaker(s) or other acoustic output device(s) of the VR goggles. Thus, the need for a dedicated medical setting or trained medical personnel is obviated.

The system is configured such that sensory filters can be trained by gradually exposing them to more difficult stimuli, as quantified by the difficulty level.

Preferably, the system further comprises an interface configured to receive a user input from the subject that triggers the control circuit to select the stimulus.

This allows the selection and outputting of the stimulus to be triggered by the subject (e.g., the wearer of VR goggles via which the stimulus is output), obviating the need for trained medical personnel. The user input may be combined with the stored difficulty levels to select the stimulus. For illustration, the stimulus may be selected such that it has a difficulty level that does not exceed a desired difficulty level specified by the user input.

Preferably, the user input specifies a desired difficulty level or a desired stimulus, wherein the control circuit is configured to use the user input to select the stimulus.

This allows the user input to be combined with the difficulty levels stored in the system to select a suitable stimulus. For illustration, the stimulus may be selected such that it has a difficulty level that does not exceed a desired difficulty level specified by the user input.

Preferably, the control circuit is configured to determine a time period for which the subject stayed exposed to the selected stimulus. The control circuit may protocol that time period in the record.

This allows the sensory filters to be gradually improved not only with regards to the difficulty level of a stimulus, but also allows the control circuit to take into account the time period for which the person suffering from hypersensitivity has successfully been exposed to the stimulus.

Preferably, the control circuit is configured to control the outputting of the selected stimulus in such a manner that not only the difficulty level but also the time period over which the stimulus is output (assuming that a termination criterion is not fulfilled) can be consistently increased. The termination criterion may be a safeguard against excessive stress and may include any one or any combination of a termination prior to a maximum time period, based on a user feedback action and/or based on a measured physiological response.

Thereby, the system is operative such that not only the difficulty level but also the exposure time can be trained in a systematic manner.

Preferably, the storage device is configured to store a record comprising difficulty levels of previously output stimuli of the plurality of stimuli, wherein the control circuit is configured to use the record to select the stimulus.

This allows the previous performance, in particular the history of difficulty levels and optionally time periods of exposure to the stimuli, to be taken into consideration. The record may be used to ensure that the difficulty level is changed only gradually, preventing jumps in the difficulty level greater than a threshold that could be detrimental to the well-being and therapeutic progress of the subject.

Preferably, the control circuit is configured to store the difficulty level of the selected stimulus in the record when causing the selected stimulus to be output.

This allows the system operation to be continually monitored and used for subsequent

Preferably, the control circuit is configured to process a physiological response of the subject to the selected stimulus to monitor a therapeutic progress. The therapeutic progress may comprise a change in stress.

This allows the physiological response of the subject to be continually monitored. The physiological response may be used by the control circuit when selecting a further stimulus in a subsequent session, based on the improvement attained when exposing the subject to the selected stimulus.

The physiological response may comprise a digital biomarker.

Thereby, digital biomarkers can be used to monitor the response of the subject during exposure to the stimulus, e.g., in an ongoing manner (i.e., continually).

Preferably, the system further comprises a physiological response measuring device and configured to measure the physiological response. The physiological response measuring device may be or may comprise a wearable. The physiological response measuring device may be different and separate from the output modality via which the selected stimulus is output.

This allows the physiological response of the subject to be monitored using a device that can be conveniently attached to the subject's body, such as a wrist-attached wearable device. It is possible but not required to use dedicated medical equipment for monitoring the physiological response.

Preferably, the system further comprises a user equipment (UE) of a cellular network configured to communicate with the physiological response measuring device and the control circuit to provide the measured physiological response to the control circuit.

This allows the physiological response to be conveyed to the control circuit using equipment available to the subject for use with the physiological response measuring device (e.g., wearable) that measures the physiological response.

Preferably, the physiological response measuring device is configured to measure one, several, or all of: a heart rate, a blood pressure, a skin temperature, a skin moisture, a skin conductance, electrodermal activity (EDA), eye pupil dilation.

Thereby, a change in stress can be detected by processing the changes in the physiological response.

Preferably, the physiological response comprises one, several, or all of: a heart rate, a blood pressure, a skin temperature, a skin moisture, a skin conductance, electrodermal activity (EDA), eye pupil dilation.

Thereby, a change in stress can be detected by processing the changes in the physiological response that occurred during the outputting of the selected stimulus.

Preferably, the system further comprises an input/output (I/O)-interface for capturing responses from the subject. The I/O-interface may be a graphical user interface (GUI) configured to capture answers of the subject to questions related to the subject's health condition, in particular related to the subject's mental condition. The control circuit may be configured to process the captured answers to determine a change in fatigue attained by the stimulus. The determined change in fatigue may be stored in the record and used for selecting another stimulus in a subsequent (e.g., next) stimulation session.

Thereby, the effects of the stimulus on the subject's fatigue may be taken into account in stimulus selection.

The record may include the change in the subject's fatigue attained by previously output stimuli, and the control circuit may use this information when selecting the stimulus.

Thereby, the effects of the stimulus on the subject's fatigue may be taken into account when selecting the stimulus.

Preferably, the control circuit is configured to cause the determined therapeutic progress to be output.

This allows the therapeutic progress to be reviewed by the subject. Alternatively or additionally, the therapeutic progress may be shared, via a wide area network, with a medical professional to whom the therapeutic progress may be transmitted via a secure transmission channel (using, e.g., authentication and/or encryption). Cloud-based storage may be implemented to allow the medical professional to access the therapeutic progress.

Preferably, the system further comprises a communication interface configured for secure transmission of data related to the outputting of the selected stimulus.

This allows data such as the therapeutic progress to be reviewed by a medical professional without requiring on-site presence of the medical professional.

Preferably, the data comprises the difficulty level of the selected stimulus and an interval length for which the subject was exposed to the selected stimulus.

This allows system usage to be reviewed by a medical professional without requiring on-site presence of the medical professional.

Preferably, the selected stimulus comprises a VR scene and the system is configured to enable the subject to navigate through the VR scene. Preferably, the VR scene comprises a 360° real-world scene.

Thereby, the sensory filters may be trained with stimuli to which the subject will be subsequently exposed in real life. By exposure to a 360° real-world scene, the sensory filters can be trained in a realistic manner for the stimuli to which the subject will be subsequently exposed in real life.

Preferably, the system comprises a computer or a game console, and the VR goggles, wherein the computer or game console comprises the control circuit and the storage device and the control circuit is configured to be communicatively coupled to the VR goggles to cause the selected stimulus to be output.

This allows the processing loads required for navigating through a 3D stimulus scene to be accommodated by the computer or game console, while implementing the VR goggles as a light-weight and convenient to use device.

Preferably, the system is a system for treating hypersensitivity of a subject with autism spectrum disorder (ASD), attention deficit hyperactivity disorder (ADHD), traumatic brain injury (TBI), stroke, fibromyalgia, post-traumatic stress disorder (PTSD), complex post-traumatic stress disorder (cPTSD), developmental trauma disorder (DTD), other mental disorders.

Thereby, the system can be configured for various medical conditions in which hypersensitivity occurs.

The difficulty levels may be dependent on the medical condition with which the subject has been diagnosed. For illustration, a first set of stimuli with a first set of difficulty levels may be available for selection when the subject has been diagnosed with a first condition selected from the group consisting of autism spectrum disorder (ASD), attention deficit hyperactivity disorder (ADHD), traumatic brain injury (TBI), stroke, fibromyalgia, post-traumatic stress disorder (PTSD), complex post-traumatic stress disorder (cPTSD), developmental trauma disorder (DTD); and a second set of stimuli with a second set of difficulty levels may be available for selection when the subject has been diagnosed with a second condition selected from this group, the second condition being different from the first condition.

The system may comprise accessory equipment operative to interact with the subject during the exposure. The accessory equipment may comprise equipment that is responsive to subject actions and/or that acts on the subject by exerting forces onto the subject or otherwise. The accessory equipment may comprise a balance board. The accessory equipment may comprise one or several actuators to apply forces onto the subject in accordance with a 360° real-world scene through which the subject navigates.

Thereby, the subject's sensory filters may be trained in a gradual manner, taking into account the subject's motions and/or forces that the subject experiences during training in accordance with the 360° real-world scene through which the subject navigates.

According to another aspect, the invention is a method of controlling a device that outputs a stimulus to the subject, the method being performed by a control circuit and comprising: accessing, by the control circuit, a storage device that stores a plurality of stimuli and difficulty levels for the plurality of stimuli; selecting, by the control circuit, a stimulus of the plurality of stimuli; and outputting, by the control circuit, control signals or control data to the device to cause the device to output the selected stimulus to the subject. The control circuit uses the stored difficulty levels to select the stimulus.

The method controls the device to output a stimulus selected in accordance with assigned difficulty levels, thereby providing an objective criterion for identifying a suitable stimulus. The method uses a suitable output modality for outputting the stimulus (such as VR goggles), and the control circuit and storage device may be provided in a personal computer (PC) or game console. Thus, the need for a dedicated medical setting or trained medical personnel is obviated.

The device to output the selected stimulus may comprise a visual output device and/or an acoustic output device of VR goggles.

The method may be performed automatically by the control circuit or system according to any aspect or embodiment disclosed herein.

Preferred additional features of the method and the effects attained thereby correspond to the features disclosed in association with the system according to the invention.

According to another aspect, the invention is a method of treating hypersensitivity of a subject, the method being performed by a control circuit and comprising: accessing, by the control circuit, a storage device that stores a plurality of stimuli and difficulty levels for the plurality of stimuli; selecting, by the control circuit, a stimulus of the plurality of stimuli; and causing, by the control circuit, the selected stimulus to be output to the subject. The control circuit uses the stored difficulty levels to select the stimulus.

The method controls the outputting of a stimulus selected in accordance with assigned difficulty levels, thereby providing an objective criterion for identifying a suitable stimulus. The method uses a suitable output modality (e.g., VR goggles), and the control circuit and storage device may be provided in a personal computer (PC) or game console. Thus, the need for a dedicated medical setting or trained medical personnel is obviated.

The stimulus may comprise a visual stimulus output via optical output device(s) of VR goggles and/or an acoustic stimulus output via acoustic output device(s) of the VR goggles.

The method may be performed automatically by the control circuit or system according to any aspect or embodiment disclosed herein.

Preferred additional features of the method and the effects attained thereby correspond to the features disclosed in association with the system according to the invention.

According to another aspect, the invention is a method of treating hypersensitivity of a subject diagnosed with autism spectrum disorder (ASD), attention deficit hyperactivity disorder (ADHD), traumatic brain injury (TBI), stroke, fibromyalgia, post-traumatic stress disorder (PTSD), complex post-traumatic stress disorder (cPTSD), developmental trauma disorder (DTD), other mental disorders, the method being performed by a control circuit and comprising: accessing, by the control circuit, a storage device that stores a plurality of stimuli and difficulty levels for the plurality of stimuli; selecting, by the control circuit, a stimulus of the plurality of stimuli; and causing, by the control circuit, the selected stimulus to be output to the subject. The control circuit uses the stored difficulty levels to select the stimulus.

The method controls the outputting of a stimulus selected in accordance with assigned difficulty levels, thereby providing an objective criterion for identifying a suitable stimulus. The method uses VR goggles as an output modality, and the control circuit and storage device may be provided in a personal computer (PC) or game console. Thus, the need for a dedicated medical setting or trained medical personnel is obviated.

The stimulus may comprise a visual stimulus output via optical output device(s) of VR goggles and/or an acoustic stimulus output via acoustic output device(s) of the VR goggles.

The method may be performed automatically by the control circuit or system according to any aspect or embodiment disclosed herein.

Preferred additional features of the method and the effects attained thereby correspond to the features disclosed in association with the system according to the invention.

The system or the method according to any aspect or embodiment disclosed herein may be configured such that the system may use subject-specific options to select the stimulus. The subject-specific options may comprise avoid options (which cause certain stimuli to be excluded from being selected by the system) and/or prefer options (which cause other stimuli to be prioritized for being selected by the system). The system or the method may be operative to control an interface (such as an HMI and/or a data interface) to enable inputting of the avoid options and/or prefer options. Alternatively or additionally, the system or the method may be operative to set or adjust the avoid options and/or prefer options based on a performance of the subject upon exposure to previous stimuli.

Thereby, subject-specific avoid and/or prioritization options may be used to take into account subject-specific limitations and needs in an efficient manner.

According to another aspect of the invention, there is disclosed machine-readable instruction code which, when executed by one or several programmable circuits (e.g., the control circuit of the system), cause execution of the method according to an aspect or embodiment of the invention.

According to another aspect of the invention, there is disclosed a non-transitory storage medium storing machine-readable instruction code which, when executed by one or several programmable circuits (e.g., the control circuit of the system), cause execution of the method according to an aspect or embodiment of the invention.

The following list of numbered embodiments defines embodiments of the invention disclosed herein:

Embodiment 1: A system for reducing hypersensitivity of a subject, comprising: a storage device configured to store a plurality of stimuli; a control circuit coupled to the storage device and configured to select a stimulus of the plurality of stimuli and cause the selected stimulus to be output to the subject; characterized in that the storage device is configured to store difficulty levels for the plurality of stimuli; and the control circuit is configured to use the stored difficulty levels to select the stimulus.

Embodiment 2: The system of embodiment 1, further comprising an interface configured to receive a user input from the subject that triggers the control circuit to select the stimulus.

Embodiment 3: The system of embodiment 2, wherein the user input specifies a desired difficulty level or a desired stimulus, wherein the control circuit is configured to use the user input to select the stimulus.

Embodiment 4: The system of any one of the preceding embodiments, wherein the storage device is configured to store a record comprising difficulty levels of previously output stimuli of the plurality of stimuli, wherein the control circuit is configured to use the record to select the stimulus.

Embodiment 5: The system of embodiment 4, wherein the control circuit is configured to store the difficulty level of the selected stimulus in the record when causing the selected stimulus to be output.

Embodiment 6: The system of any one of the preceding embodiments, wherein the control circuit is configured to process a physiological response of the subject to the selected stimulus to monitor a therapeutic progress.

Embodiment 7: The system of embodiment 6, further comprising a physiological response measuring device configured to measure the physiological response.

Embodiment 8: The system of embodiment 7, further comprising a user equipment, UE, of a cellular network configured to communicate with the physiological response measuring device and the control circuit to provide the measured physiological response to the control circuit.

Embodiment 9: The system of any one of embodiments 6 to 8, wherein the physiological response comprises one, several, or all of: a heart rate, a blood pressure, a skin temperature, a skin moisture, a skin conductance, electrodermal activity (EDA), eye pupil dilation.

Embodiment 10: The system of any one of embodiments 6 to 9, wherein the control circuit is configured to at least one of: cause the determined therapeutic progress to be output; cause outputting of the selected stimulus to be terminated in response to detecting from the physiological response that the stimulus causes an unacceptable stress level for the subject.

Embodiment 11: The system of any one of the preceding embodiments, further comprising a communication interface configured for secure transmission of data related to the outputting of the selected stimulus, optionally wherein the data comprises the difficulty level of the selected stimulus and an interval length for which the subject was exposed to the selected stimulus.

Embodiment 12: The system of any one of the preceding embodiments, wherein the selected stimulus comprises a VR scene and the system is configured to enable the subject to navigate through the VR scene, optionally wherein the VR scene comprises a 360° real-world scene.

Embodiment 13: The system of any one of the preceding embodiments, wherein the system comprises a computer or a game console, and VR goggles comprising an optical output device and an acoustic output device, wherein the computer or game console comprises the control circuit and the storage device and the control circuit is configured to be communicatively coupled to the VR goggles to cause the selected stimulus to be output via the optical output device and/or the acoustic output device.

Embodiment 14: The system of any one of the preceding embodiments, wherein the system is a system for treating hypersensitivity of a subject with autism spectrum disorder (ASD), attention deficit hyperactivity disorder (ADHD), traumatic brain injury (TBI), stroke, fibromyalgia, post-traumatic stress disorder (PTSD), complex post-traumatic stress disorder (cPTSD), developmental trauma disorder (DTD), other mental disorders.

Embodiment 15: A method of controlling a device that outputs a stimulus to a subject, the method being performed by a control circuit and comprising: accessing, by the control circuit, a storage device that stores a plurality of stimuli and difficulty levels for the plurality of stimuli; selecting, by the control circuit, a stimulus of the plurality of stimuli; and outputting, by the control circuit, control signals or control data to the device that cause the device to output the selected stimulus to the subject; wherein the control circuit uses the stored difficulty levels to select the stimulus.

### BRIEF DESCRIPTION OF THE DRAWINGS

The system and method according to the invention are described in more detail below by way of an exemplary embodiment and with reference to the attached drawings, in which:

FIG. 1 shows a system for reducing hypersensitivity in accordance with an embodiment;

FIG. 2 shows a flow chart of a method according to an embodiment;

FIG. 3 shows a system for reducing hypersensitivity of FIG. 1 in association with additional components;

FIG. 4 shows a flow chart of a method according to an embodiment;

FIG. 5 shows a flow chart of a method according to an embodiment;

FIG. 6 shows the system of FIG. 3 with additional accessory equipment; and

FIG. 7 shows a flow chart of a method according to an embodiment.

### DESCRIPTION OF EMBODIMENTS

In the following description certain terms are used for reasons of convenience and are not intended to limit the invention. The terms “right”, “left”, “up”, “down”, “under” and “above” refer to directions in the figures. The terminology comprises the explicitly mentioned terms as well as their derivations and terms with a similar meaning. Also, spatially relative terms, such as “beneath”, “below”, “lower”, “above”, “upper”, “proximal”, “distal”, and the like, may be used to describe one element's or feature's relationship to another element or feature as illustrated in the figures. These spatially relative terms are intended to encompass different positions and orientations of the devices in use or operation in addition to the position and orientation shown in the figures. For example, if a device in the figures is turned over, elements described as “below” or “beneath” other elements or features would then be “above” or “over” the other elements or features. Thus, the exemplary term “below” can encompass both positions and orientations of above and below. The devices may be otherwise oriented (rotated 90 degrees or at other orientations), and the spatially relative descriptors used herein interpreted accordingly. Likewise, descriptions of movement along and around various axes include various special device positions and orientations.

To avoid repetition in the figures and the descriptions of the various aspects and illustrative embodiments, it should be understood that many features are common to many aspects and embodiments. Omission of an aspect from a description or figure does not imply that the aspect is missing from embodiments that incorporate that aspect. Instead, the aspect may have been omitted for clarity and to avoid prolix description. In this context, the following applies to the rest of this description: If, in order to clarify the drawings, a figure contains reference signs which are not explained in the directly associated part of the description, then it is referred to previous or following description sections. Further, for reason of lucidity, if in a drawing not all features of a part are provided with reference signs it is referred to other drawings showing the same part. Like numbers in two or more figures represent the same or similar elements.

In the following, systems and methods can be used that allow hypersensitivity to be reduced. As used herein, the term “hypersensitivity” is a sensory processing disorder. The term “hypersensitivity” encompasses impaired sensory filtering. Any reference to “hypersensitivity” can be replaced by a reference to “impaired sensory filtering” and vice versa within this disclosure.

The term “hypersensitivity” is a generic term for allergy, pseudo allergy, intolerance and idiosyncrasy. In that case the perception of the affected people is not filtered which can result in a sensory overload. Sensory overload occurs when one or more of the body's senses experiences overstimulation from the environment.

The systems and methods disclosed herein are configured to train the visual and/or auditory sensory filters, to thereby reduce visual and/or auditory hypersensitivity.

This is done by exposing the subject suffering from hypersensitivity to stimuli in a controlled environment. The subject builds his/her sensory filters from the subconsciousness by training the visual and acoustic filters.

The systems and methods allow a difficulty level and, optionally, exposure time to be gradually increased in a controlled environment. The difficulty level and the time spent in each level can be slowly (e.g., gradually) increased. The goal of the controlled exposure to stimuli of increasing difficulty and/or exposure time is that the sensory filters are built from the subconsciousness by training the visual and acoustic filters. This training can be implemented such that it allows the subject, after reaching a high difficulty level, to participate in the daily live and situations which caused an overstimulation before. Thus, the systems and methods disclosed herein are useful for training sensory filters which allow the subject to participate inside these situations. Therefore hypersensitivity/sensory processing disorder, with a focus on visual and acoustic stimuli, is a symptom which can be treated by this solution.

The difficulty level may respectively be a difficulty level of an ordinal scale (e.g., a level from an integer value range) or any other designation that allows the system to establish which of several stimuli is more challenging. An assignment of such difficulty levels to scenarios such as 360° real-world scenarios may be done using any conventional technique, such as in an expert-based manner (with the assignment being not part of the claimed methods) and/or in a computerized manner by automatically identifying features in the 360° real-world scenarios for use in rating the difficulty (by means of the difficulty level).

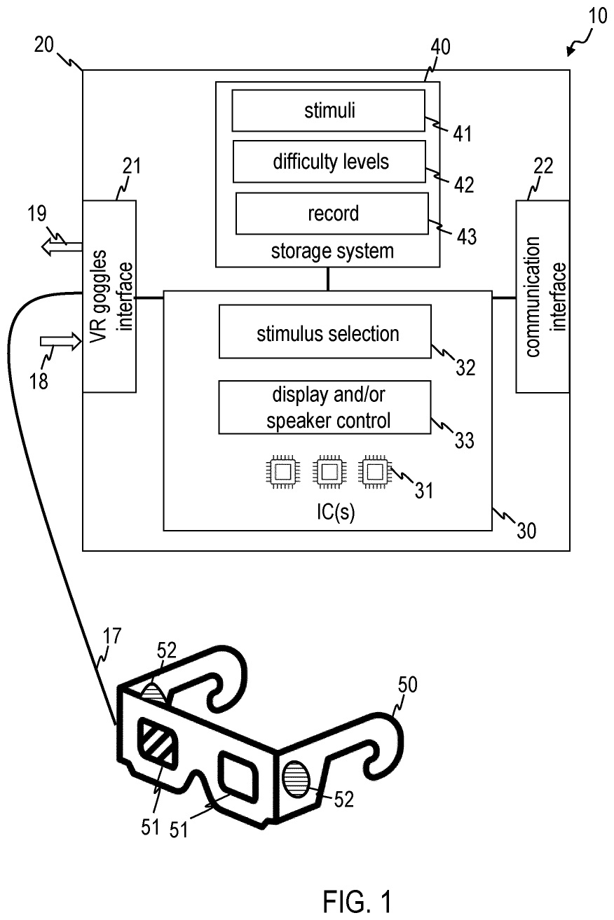

FIG. 1 shows a block diagram of a system 10 for reducing hypersensitivity according to an embodiment. The system 10 comprises virtual reality (VR) goggles 50 through which stimuli are output to a wearer.

The system 10 comprises VR goggles 50 with a pair of displays 51 through which visual stimulation occurs and electroacoustic transducers (e.g., speakers) 52 through which aural stimulation occurs. A user interface that allows the wearer of the VR goggles to provide user input may be implemented in hardware (e.g., as a joystick, mouse, controller, or smartphone) or as machine-readable instruction code (e.g., as a virtual control that can be manipulated using the VR goggles). The system 10 presents stimuli in which one or more sensory types of perception (such as only visual, only aural, or both visual and aural) are stimulated.

The system 10 and method can be used for therapeutic treatment of people with any of the following diagnoses: autism spectrum disorder (ASD), attention deficit hyperactivity disorder (ADHD), traumatic Brain Injury (TBI), stroke, fibromyalgia, post-traumatic stress disorder (PTSD), complex post-traumatic stress disorder (cPTSD), developmental trauma disorder (DTD), other mental disorders. People with this diagnosis may be affected by hypersensitivity. While the system 10 and method disclosed herein may be used with subjects diagnosed with any of these medical conditions, the system 10 and method may generally be employed for training one or several sensory filters of a subject suffering from hypersensitivity.

The system 10 comprises a computing device 20. The computing device 20 may be a personal computer or game console, without being limited thereto. The computing device 20 comprises a control circuit 30 and a storage device 40. The control circuit 30 may comprise a plurality of integrated circuit(s) (IC(s)) 31. The IC(s) may comprise one or several application specific integrated circuit(s) (ASIC(s)), controller(s), processor(s), field programmable gate array(s) (FPGA(s)), quantum gates, or combinations thereof. The control circuit 30 may execute machine-readable instruction code to perform any of the functions disclosed herein.

The storage device 40 may be a non-volatile storage device. The storage device 40 stores a plurality of stimuli 41 and a difficulty level 42 assigned to each of the plurality of stimuli. The storage device 40 may store a record 43 indicating previous operation of the computing device 40. For illustration, the difficulty levels of previously output stimuli and, optionally, the time periods spent by the user exposed to the respective stimulus may be stored in the record 43.

The computing device 20 may be coupled to the VR goggles 50 via a wired or wireless communication link 17. User input 18, which may include a selection of a stimulus or difficulty level, may be received via the link 17 at a VR goggles interface 21 of the computing device 20. Control data or signals 19 may be output via the VR goggles interface 21 over link 17 to the VR goggles 50 to control one or several displays 51 and/or one or several electroacoustic transducers 52 (e.g., speakers) to output a selected stimulus.

The computing device 20 may have one or several additional communication interface(s) 22. The communication interface(s) 22 may be configured to receive a physiological response of the wearer of the VR goggles and/or for transmitting data indicating a therapeutic progress via a secured communication channel.

The physiological response may be recorded in the record 43 and used when selecting a next stimulus. Vice versa, previously recorded physiological responses may be retrieved from the record 43 and used by the control circuit 30 when selecting the stimulus. The physiological response may also be used to determine whether the outputting of a stimulus is to be terminated, e.g., when detecting an excessive stress (e.g., a meltdown situation) by processing the physiological response. This will be described in more detail with reference to FIG. 5.

The physiological response may be received from a physiological response measuring device. Alternatively or additionally, the physiological response may include a fatigue derived from the subject's answers to health-related questions, which may be enabled by controlling a graphical user interface (GUI) to allow the inputting of these answers.

During operation, the control circuit 30 executes a stimulus selection 32 to select a stimulus for outputting. The selection may be triggered by the user input 18 specifying a desired difficulty level. The control circuit 30 may use the stored difficulty levels 42, the difficulty level indicated by the user input 18, and the difficulty levels previously recorded in the record 43 to select a suitable stimulus. For illustration, the selection may be done in such a way that stimuli of monotonously (but not necessarily strictly monotonously) increase difficulty level are output one after the other, while preventing changes in difficulty level between successive sessions that exceed a threshold.

During operation, the control circuit 30 executes a display and/or speaker control 33 to cause the displays 51 of the VR goggles 50 to display a visual stimulation included in the selected stimulus and/or to cause the speakers 52 of the VR goggles 50 to play a sound included in the selected stimulus.

The system 10 enables a subject to select different levels that will be displayed on the VR goggles 50 and sound from the levels will be played on the speakers 52 from the VR goggles 50. The levels may be separated and marked by the sensory type. Sensory types or so-called sensory categories that may be stimulated are: Visual and acoustic. The stimuli can include stimuli with difficulty levels that focus on one sensory type or combine both.

Increasing difficulty levels result in more sensory stimulus. A higher stimulus in that case results in more acoustic signal sources and/or more visual inputs. For example a train station with many people walking, talking, trains arriving and departing has a higher difficulty level.

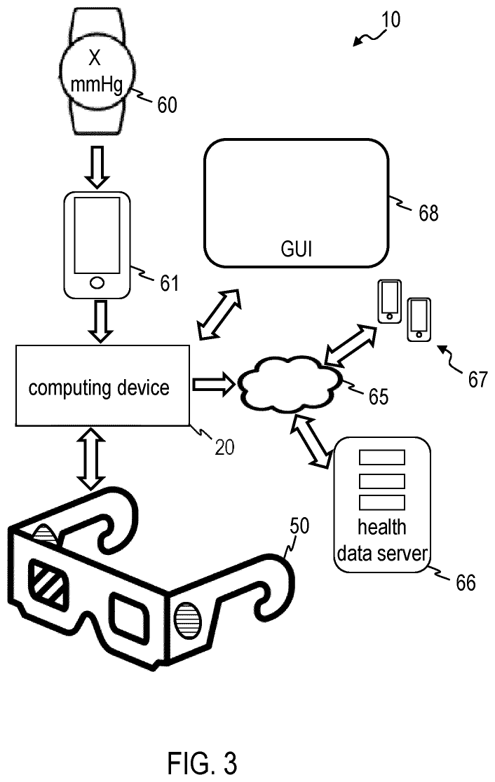

The system 10 enables a subject to select one difficulty level and stop it after some time was spent inside the difficulty level. The time spent in this difficulty level is protocolled by the computing device 20 by storing the time in the record 43. Optionally, the evolution of a physiological response (which may include one, several, or all of: a heart rate, a blood pressure, a skin temperature, a skin moisture, a skin conductance,, electrodermal activity (EDA), eye pupil dilation) while the subject is being exposed to the selected stimulus having the selected difficulty level may be protocolled by the computing device 20 by storing the time in the record 43. The tracking of the physiological parameter is optional and will be described in more detail with reference to FIG. 3.

Eye pupil dilatation can be measured using a camera included in the VR goggles and directed towards the user's eyes.

Stress is determined: from operative measurements plus subjective response.

Additionally or alternatively to monitoring the physiological response using the physiological response measuring device, the computing device 20 may collect the subject's answers to questions to determine a change in fatigue level. The system 10 may further comprise an input/output (I/O)-interface for capturing the answers from the subject. The I/O-interface may be a graphical user interface (GUI) configured to capture answers of the subject to questions related to the subject's health condition, in particular related to the subject's mental condition. The control circuit 30 may be configured to process the captured answers to determine a change in fatigue attained by the stimulus. The determined change in fatigue may be stored in the record 43 and used for selecting another stimulus in a subsequent (e.g., next) stimulation session. Vice versa, previously recorded changes in fatigue level may be retrieved from the record 43 and used for selecting the stimulus (e.g., by using information on the difficulty levels of previously output stimuli that affected the user's sensory filters in a beneficial way).

The goal is for the wearer of the VR goggles 50 to be able to stay longer inside the selected difficulty level with every training and experience less stress and elevated pulse while being exposed to the stimulus. The wearer of the VR goggles 50 might feel uncomfortable and be somewhat overstimulated after ending a level but shall not experience overstimulation. In case of overstimulation, the wearer of the VR goggles 50 was in the selected level for too long. People with ASD shall not experience overstimulation, which is called meltdown in that case.

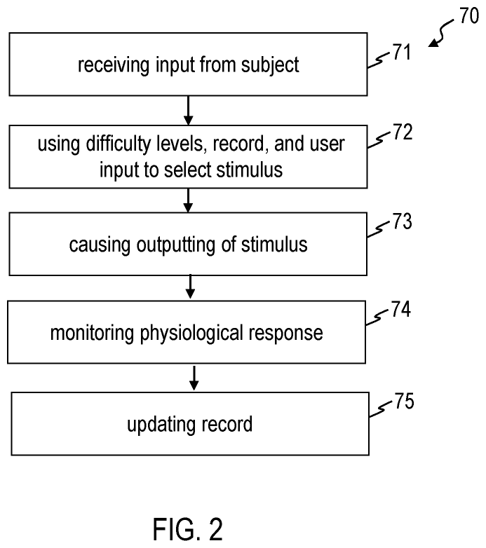

FIG. 2 is a flow chart of a method 70. The method 70 may be performed automatically be the system 10, e.g., by the control circuit 30 of the system 10.

At step 71, a user input 18 is received. The user input 18 may indicate a desired difficulty level.

At step 72, a stimulus is selected. To select the stimulus, the control circuit accesses the storage device 40 that stores the plurality of stimuli 41 and the difficulty levels 42 for the plurality of stimuli. The control circuit 30 may select the stimulus such that it is consistent with the subject's indication (e.g., more challenging than previous difficulty levels), the previously completed stimuli and their difficulty levels, and prevents too abrupt changes in difficulty level.

Additional information may be taken into account by the control circuit 30 when selecting the stimulus to be output. For illustration, the record 43 may store physiological responses to previous stimuli. The control circuit 30 may be configured to determine difficulty levels of previously output stimuli that caused increased stress, as measured by a physiological response, during exposure, but which did not cause an excessive stress (based on, e.g., a threshold comparison of one or several physiological parameters such as heart rate, blood pressure, skin temperature, electrodermal activity (EDA), skin conductance, eye pupil dilatation etc.). The stimulus may be selected such that it has a higher difficulty level than a previously output stimulus that caused increased stress without causing excessive stress.

Alternatively or additionally, information on the time period spent in previously output stimuli may be retrieved from the record 43. The control circuit 30 may be configured to determine difficulty levels of previously output stimuli that the subject was able to withstand for a maximum time period (which may be configurable and pre-configured and which may be on the order of, e.g., up to 1 hour, up to 2 hours, etc.). The control circuit 30 may be configured to select the stimulus such that it has a higher difficulty level than a previously output stimulus that the subject was able to withstand without early termination prior to the maximum time period. The information on the previous time period may be used to set a maximum time period for which the selected stimulus will be output.

The stimulus may comprise a 360° real-world image or video sequence through which the wearer of the VR goggles 50 can navigate and sound associated with the real-world image or video sequence. The 360° real-world image or video sequence comprises images and/or videos that are recorded from real-world scenarios and that are not rendered during outputting of the stimulus. Using 360° real-world image or video sequences provides more detailed visual and, optionally, acoustic stimuli as compared to stimuli that are synthetically generated by rendering. Thereby, a better training of the sensory filters is attained as compared to exposure to stimuli generated by rendering. Alternatively or additionally to visual and/or aural stimulation, the selected stimulus may provide tactile and/or olfactory stimulation.

At step 73, the control circuit 30 causes the selected stimulus to be output to a wearer of virtual reality (VR) goggles via the visual output device 51 and/or the aural output device 52 of the VR goggles 50. Alternatively or additionally to visual and/or aural stimulation, the selected stimulus may provide tactile and/or olfactory stimulation.

At step 74, the control circuit 30 monitors a physiological response of the wearer of the VR goggles 50 while he/she is being exposed to the selected stimulus. The monitoring may comprise monitoring a physiological parameter (such as heart rate and/or blood pressure or other parameters such as a skin temperature, a skin moisture, a skin conductance, electrodermal activity (EDA), eye pupil dilation). The monitoring may comprise assessing a change in sensory overload experienced during exposure to the selected stimulus. The monitoring may comprise assessing a change in stress experienced during exposure to the selected stimulus. Alternatively or additionally to the measurement of physiological responses during outputting of the selected stimulus, the system 10 may be configured to control a GUI to enable user input that is processed by the control circuit 30 to assess fatigue or a change in fatigue caused by the outputting of the selected stimulus. To enable the user input, the system 10 may be configured to control the GUI to output questions to the subject and receive the subject's answers for processing by the control circuit 30. The system 10 may be configured to control the GUI to enable a selection from among various choices (e.g., in the form of a drop-down menu, a slider or dial that can be manipulated to indicate a user input, toggle switches, or other input elements) to receive the user input.

At step 75, the control circuit updates the record 43. Updating the record may comprise adding information on the stimulus (in particular its difficulty level) and time period which the subject stayed exposed to the stimulus. This information may be time-stamped, e.g., indicating the date on which the wearer of the VR goggles 50 was exposed to the stimulus. Updating the record 43 may comprise storing information on the physiological response in the record 43. Updating the record 43 may comprise storing information on the time period spent by the subject while being exposed to the stimulus in the record 43.

FIG. 3 shows a block diagram of a system 10 that comprises components in addition to the computing device 20 and VR goggles 50.

For monitoring a physiological response, the computing device 20 may be configured to be communicatively coupled to a physiological response measuring device. The physiological response measuring device may be a wearable 60. The wearable 60 may be a wrist-attachable wearable. The wearable 60 may be configured to measure a physiological response. The physiological response may comprise one, several, or all of: a heart rate, a blood pressure, a skin temperature, a skin moisture, a skin conductance, electrodermal activity (EDA), eye pupil dilation. The physiological response may also be used in a safety mechanism which determines whether the selected stimulus is to be terminated early by the control circuit 30, as will be described in more detail with reference to FIG. 5.

Alternatively or additionally to using the wearable 60 or other measuring device that captures the physiological response by measurement, the computing device 20 may collect the subject's answers to questions to determine a change in fatigue level. The system 10 may have a GUI 68. The computing device 20 may be configured to control the GUI 68 to enable inputting of answers of the subject to questions related to the subject's health condition, in particular related to the subject's mental condition. The control circuit 30 may be configured to process the captured answers to determine a change in fatigue. The determined change in fatigue may be stored in the record 43 and used for selecting another stimulus in a subsequent (e.g., next) stimulation session. Vice versa, previously recorded changes in fatigue level may be retrieved from the record 43 and used for selecting the stimulus (e.g., by using information on the difficulty levels of previously output stimuli that affected the user's sensory filters in a beneficial way).

For ease of implementation, the computing device 20 may be communicatively coupled to the physiological response measuring device (e.g., wearable) 60 via a user equipment (UE) 61 of a cellular network, such as a smartphone. This is particularly convenient when the physiological response measuring device is a wearable that is configured to be communicatively coupled to the UE 61 for, e.g., transferring measurements to the UE 61.

The computing device 20 may share information collected for the wearer of the VR goggles 50. For illustration, the computing device 20 may be configured to transmit monitored usage data (such as the selected difficulty level, time spent by the subject exposed to the stimulus, and date of use) via a wide area network (WAN) (such as the internet) 65 to a health data server 66. The computing device 20 may be configured to transmit a monitored physiological response (such as the change in heart rate, blood pressure, skin temperature, skin moisture, skin conductance, electrodermal activity (EDA), eye pupil dilation and/or other physiological parameters monitored for the wearer of the VR goggles 50 while he/she was navigating through a stimulus). The data may be stored in a subject-specific storage space of the health data server 66.

The data may be accessed by medical professional using their terminal devices 67. For illustration, a medical professional may check the usage of the system 10 and/or the physiological response by accessing the subject-specific storage space of the health data server 66, when authorized to do so.

Sharing data captured by the computing device 20 over a WAN is technically beneficial because it allows medical professionals to review the subject's progress and sensory training activity, while obviating the need for the medical professionals to be present on site in each session. This reduces stress for the subject. This also reduces resources (such as fuel consumption) spent otherwise if the medical professional were to be present on site more frequently during the training sessions.

Sharing data in a cloud system in a secure manner also allows the progress and training activity to be stored for subsequent use by the subject, the subject's doctor, or other parties that may have a legitimate interest in using the data for the subject's medical benefit.

Safety mechanisms employed by the components of the system 10 (such as the computing system 20) safeguard the privacy of the data.

Authentication techniques, such as two-factor authentication (2FA), may be used for accessing the subject-specific storage space on the health data server 66. All data transmission may be encrypted. Other safety techniques may be used to safeguard the privacy of sensitive subject-specific information.

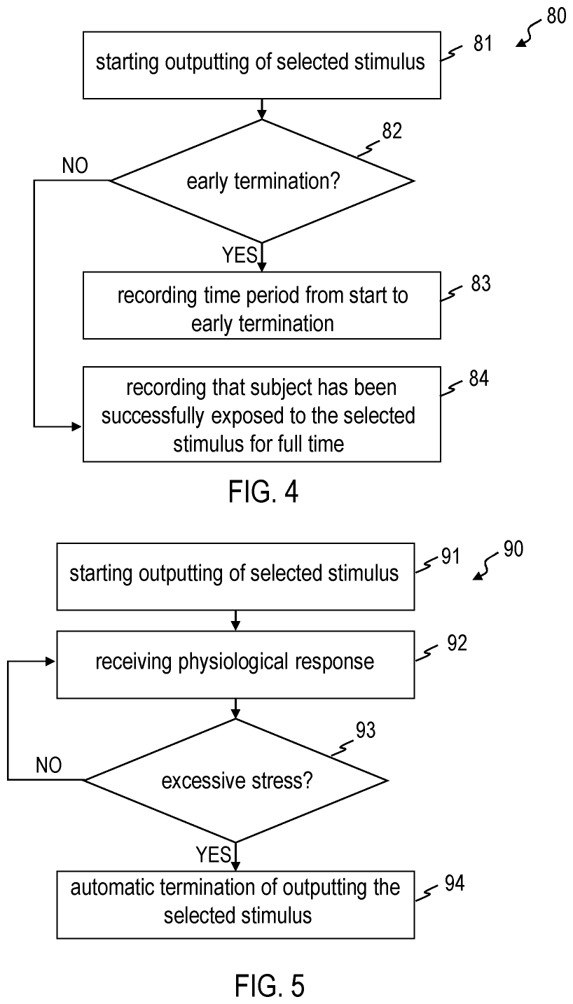

FIG. 4 is a flow chart of a method 80. The method 80 may be performed automatically be the system 10, e.g., by the control circuit 30 of the system 10.

At step 81, the control circuit 30 causes outputting of the selected stimulus to be started. Selection of the stimulus may be implemented in any of the ways described above.

At step 82, it is determined whether the subject has terminated exposure to the stimulus early. For illustration, the computing device 20 may be configured to receive and process data from the VR goggles 50 indicating that the user has pressed a stop button or has removed the VR goggles 50.

At step 83, if early termination is detected, the time period spent by the subject exposed to the stimulus may be recorded in the record 43. Pauses, if any, may be accounted for when determining the time period spent exposed to the stimulus.

At step 84, if the subject does not terminate the exposure to the stimulus early, the record 43 is updated to indicate this fact in association with the difficulty level. For illustration, the time for which the stimulus was output and/or an indicator indicating that the maximum exposure time was reached may be stored in the record.

The information on the time spend in a stimulus may be used by the control circuit 30 when selecting the next stimulus, as previously explained.

A physiological response to the stimulus may be monitored for assessing the therapeutic progress and for use in the selection of the next stimulus. Additionally or alternatively, the physiological response may be monitored and used in a safety mechanism that prevents the subject from undergoing excessive stress, as will be described in association with FIG. 5.

FIG. 5 is a flow chart of a method 90. The method 90 may be performed automatically be the system 10, e.g., by the control circuit 30 of the system 10.

At step 91, the control circuit 30 causes outputting of the selected stimulus to be started. Selection of the stimulus may be implemented in any of the ways described above.

At step 92, a physiological response is captured. Capturing the physiological response may comprise receiving and processing measurements from a physiological response measuring device 60.

At step 93, it is determined whether the subject is presently experiencing excessive stress. The determination at step 93 is made while the subject is still exposed to the selected stimulus. The determination at step 93 may be made in real time. The determination at step 93 may be continually updated during the subject's exposure to the selected stimulus. The determination at step 93 may comprise performing a threshold comparison for at least one physiological parameter measured on the subject. For illustration, the subject's heart rate, blood pressure, skin temperature, skin conductivity, EDA, and/or eye pupil dilation or other physiological parameter(s), may be compared to an associated threshold to detect excessive stress. Alternatively or additionally, the change or rate of change of the subject's heart rate, blood pressure, skin temperature, skin conductivity, EDA, and/or eye pupil dilation or other physiological parameter(s), may be compared to an associated threshold to detect excessive stress. Filtering techniques may be applied to the measured physiological parameter(s) prior to the thresholding, to eliminate outliers and/or false stress detection results.

If no excessive stress is detected at step 93, the monitoring at steps 92 and 93 is repeated. The monitoring for excessive stress may be repeated in an ongoing basis, e.g. continually.

At step 94, if excessive stress is detected at step 93, the outputting of the stimulus is actively terminated by the control circuit 30. For illustration, a control signal or control data may be output to the output modality of the selected stimulus (e.g., the display(s) 51 and/or speakers 52 of the VR goggles 50) to terminate the exposure to the stressful situation.

The process disclosed in FIG. 5 is generally beneficial for safety reasons and is particularly useful to prevent meltdown situations in, e.g., subjects diagnosed with ASD.

Information on the termination at step 94 may be stored in the record 43, in association with the difficulty level, the time spent by the subject exposed to the stimulus, and the stress level (e.g., physiological parameter identified as being indicative of excessive stress). This information may be retrieved and used when selecting the next stimulus in a subsequent session.

In any one of the embodiments disclosed herein, the system may be configured such that the user can terminate or pause the exposure to a stimulus at any time.

While the systems and methods disclosed herein are configured such that they can be performed in a residential setting by the subject, without requiring the presence of a medical professional, it is preferred that the subject be trained adequately prior to using the systems and methods.

For illustration, it is preferred that the subject is being trained on using the system in the presence of a medical professional, e.g., in a hospital or doctor's office, a number of times (e.g., 4 to 8 times). It is preferred that the subject starts using the system in a home setting only after having received this training.

Safety mechanisms of the system 10 may be configured to prevent the subject from using the system 10 without presence of a medical professional unless proper training is confirmed by the medical professional. For illustration, the computing device 20 may include a safety mechanism, which may be implemented in hardware or in machine-readable instruction code, locks the initiation of a stimulation session until a medical professional releases this function for use by the subject.

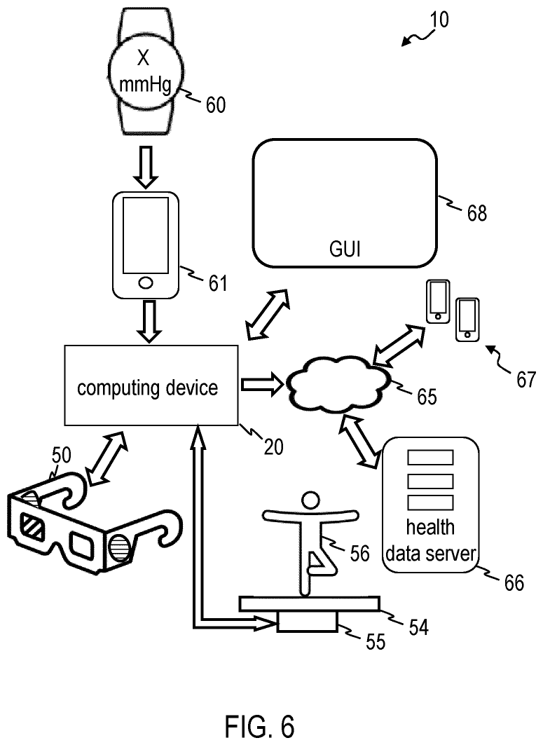

FIG. 6 shows the system 10 according to a further embodiment which comprises accessory equipment. The accessory equipment accessory equipment may be configured to interact with the subject 56 during exposure to the selected stimulus. The accessory equipment may comprise equipment that is responsive to subject actions and/or that acts on the subject by exerting forces onto the subject or otherwise. The accessory equipment may comprise a balance board 54. The balance board may optionally comprise one or several actuators 55 to apply forces onto the subject 56, e.g., in accordance with a 360° real-world scene through which the subject 56 navigates. Thereby, the subject's sensory filters may be trained in a gradual manner, taking into account the subject's motions and/or forces that the subject 56 experiences during training in accordance with the 360° real-world scene through which the subject navigates.

The actuator(s) 55 may be communicatively coupled to the computing device 20. The computing device 20 may be operative to control (e.g., by means of control data and/or control signals) the actuator(s) 55 in accordance with the selected stimulus. Thereby, the subject 56 can be exposed to forces that depend on a history of previous stimuli and the exposure time for these stimuli to which the subject 56 was previously exposed.

The actuator(s) 55 may be coupled to the computing device 20 via a bidirectional link. This allows the computing device 20 to receive from the actuator(s) 55 data or signals useful in monitoring the subject's physiological response (in particular the subject's bodily movements) during exposure to the selected stimulus.

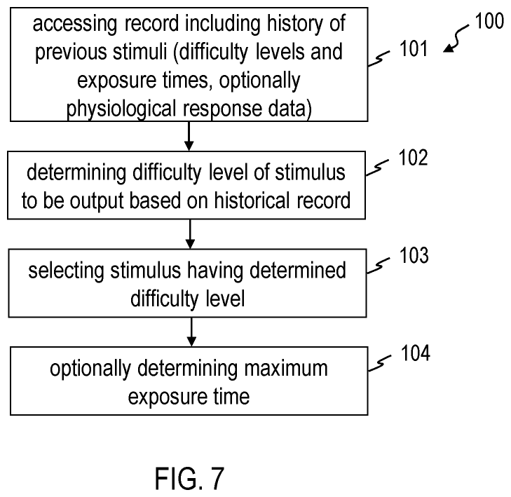

FIG. 7 is a flow chart of a method 100. The method 100 may be performed automatically be the system 10, e.g., by the control circuit 30 of the system 10. The method 100 may be performed automatically to implement step 72 of the method 70 of FIG. 2. The method 100 may be performed to utilize a history of difficulty levels, exposure times, and, optionally, physiological responses for stimuli previously output to the respective subject, in order to determine which stimuli can be output next to the subject and, optionally, for determining a maximum exposure time for which the stimulus will be output. The method 100 may be performed in particular to determine which difficulty level a stimulus to be output is to have, based on the history of difficulty levels, exposure times, and, optionally, physiological responses for stimuli previously output to the respective subject.

At step 101, the control circuit 30 accesses the record 43 which includes a history of previous stimulus output operations, including at least data representing the difficulty level and exposure time for stimuli previously output to the same subject. The record 43 may also include physiological response data, such as data indicating a stress level, as previously explained.

At step 102, the control circuit 30 determines a difficulty level of the stimulus to be output next. The control circuit 30 may be operative to determine the difficulty level to be (i) obtained by incrementing the most challenging difficulty level in the history stored in the record 43 if a difficulty level advancement criterion is fulfilled, and (ii) be equal to the most challenging difficulty level in the history stored in the record 43 if the difficulty level advancement criterion is not fulfilled. The difficulty level advancement criterion may comprise a verification that one or several of the following apply: (a) several stimuli of the most challenging difficulty level in the history stored in the record were already output to the subject; (b) the exposure time respectively was in accordance with a time threshold criterion (e.g., reaching the maximum exposure time without early termination); (c) optionally the subject's physiological response shows that the subject did not experience stress considered to be not acceptable when being exposed to these previous stimuli.

At step 103, a stimulus is selected from among the stored stimuli, the selected stimulus comprising a 360° real-world VR scene and having the difficulty level determined at step 102.

At step 104, a maximum exposure time may optionally be determined. Determining the exposure time may be performed based on the exposure times recorded in record 43 for previously output stimuli (in particular previously output stimuli of a same difficulty level or of a lower difficulty level). Thereby, the exposure time can be increased in, e.g., a gradual manner to train the subject's sensory filters in a gradual manner, both with regard to difficulty level and exposure time.

While not shown in FIG. 7, the subject's response during outputting of the selected stimulus may be monitored and the record 43 is updated for a next iteration of exposing the subject to gradually more challenging stimuli.

Various effects and advantages are attained by a system and method as disclosed herein and, in particular, by a system and method as recited in the independent claims. This will be illustrated with reference to the patent documents U.S. Pat. No. 10,809,796 B2, US 2016/275805 A1, US 2020/356136 A1, US 2020/234827 A1, and CN 110 891 638 A already mentioned in the background section.

CN 110 891 638 A discloses a system that is useful for training a physician's skills using VR techniques. CN 110 891 638 A does not disclose techniques that allow sensory filters to be improved by gradually exposing them to more difficult stimuli. CN 110 891 638 A1 does not disclose a system or method in which both difficulty levels for a plurality of stimuli and a record of historical performance (namely previously output stimuli and the respective time periods of exposure) are used to select the stimulus to be output, with the selected stimulus comprising a 360° real-world scene. Thus, the techniques disclosed in this application and, in particular, the techniques as claimed provide the effect of implementing a techniques that allows stimuli to be selected in a systematic manner to expose the subject to gradually more challenging stimuli.

U.S. Pat. No. 10,809,796 B2, US 2016/275805 A1, US 2020/356136 A1, and US 2020/234827 A1 all disclose systems that allow stimuli to be output to a user and that may monitor a response using sensors and/or user feedback. However, these systems are not configured such that both difficulty levels for a plurality of stimuli and a record of historical performance (namely previously output stimuli and the respective time periods of exposure) are used to select the stimulus to be output, with the selected stimulus comprising a 360° real-world scene. For illustration, neither one of these documents discloses that a storage device stores difficulty levels for a plurality of stimuli. While US 2016/275805 A1 discusses an adjustment of difficulty, US 2016/275805 A1 does not disclose that difficulty levels for a plurality of stimuli are used to perform this adjustment, much less that difficulty levels for a plurality of stimuli and a record of historical performance (namely previously output stimuli and the respective time periods of exposure) are used to select the stimulus to be output. While US 2016/275805 A1 discusses that an adjustment may comprise a change in duration for which sensors information is provided to an individual, this document does not disclose that time periods of exposure for previously output stimuli are stored in a record and used to select a stimulus. The other documents similarly do not disclose that both difficulty levels for a plurality of stimuli and a record of historical performance (namely previously output stimuli and the respective time periods of exposure) are used to select the stimulus to be output, with the selected stimulus comprising a 360° real-world scene. U.S. Pat. No. 10,809,796 B2 does not provide a systematic way to select stimuli that takes into account difficulty and duration of stimuli being provided. US 2016/275805 A1, US 2020/356136 A1, and US 2020/234827 A1 provide techniques that may be useful to restore lost sensory skills. The present application provides techniques that assist people struggling with too many stimuli and having difficulties in filtering them correctly. The present application provides a system and method that uses a systematic selection of stimuli so as to allow stimuli of multiple, increasing difficulty levels to be output. Thus, the techniques disclosed in this application and, in particular, the techniques as claimed provide the effect of implementing a techniques that allows stimuli to be selected in a systematic manner to expose the subject to gradually more challenging stimuli.

This description and the accompanying drawings that illustrate aspects and embodiments of the present invention should not be taken as limiting-the claims defining the protected invention. In other words, while the invention has been illustrated and described in detail in the drawings and foregoing description, such illustration and description are to be considered illustrative or exemplary and not restrictive. Various mechanical, compositional, structural, electrical, and operational changes may be made without departing from the spirit and scope of this description and the claims. In some instances, well-known circuits, structures and techniques have not been shown in detail in order not to obscure the invention. Thus, it will be understood that changes and modifications may be made by those of ordinary skill within the scope and spirit of the following claims. In particular, the present invention covers further embodiments with any combination of features from different embodiments described above and below. For example, it is possible to operate the invention in an embodiment or in embodiments wherein on, several, or all of the following apply:
the control circuit 30 and optionally the storage device 40 are integrated with the VR goggles 50,
the heart rate and blood pressure are not monitored,
physiological parameters other than heart rate and blood pressure are monitored in addition to or as an alternative to monitoring heart rate and blood pressure, such as eye pupil dilation and/or EDA,
additional criteria are taken into account when selecting a stimulus, in addition to considering the difficulty level,
the stimuli act on senses other than visual and/or auditory senses; for illustration, the selected stimulus (and several of the plurality of stimuli) may comprise tactile stimuli and/or olfactory stimuli, in addition to or as an alternative to visual and/or aural stimuli;
the output device(s) for outputting the selected stimulus does not comprise VR goggles 50 but comprises other output modalities, such as an olfactory stimulus generation device and/or a piece of clothing with actuators configured to provide tactile stimuli.

In all embodiments, the difficulty levels may be pre-determined using expert knowledge and/or prior testing. Alternatively or additionally, the difficulty levels may be pre-determined by the computer system in an automated manner, with the processing system being operative to perform object recognition and/or other image processing techniques in the VR scenes (e.g., the 360° VR scenes) to determine the difficulty level for each of the stimuli. The difficulty level may be assigned automatically based on any one or any combination of, e.g., the following: a number of people or objects in the VR scene, a number of moving people and/or moving objects in the scene, a number of people and/or objects in the scene that move at a speed in excess of a threshold speed, brightness level, changes in brightness level, audio signal level, changes in audio signal level.

The disclosure also covers all further features shown in the Figs. individually although they may not have been described in the afore or following description. Also, single alternatives of the embodiments described in the figures and the description and single alternatives of features thereof can be disclaimed from the subject matter of the invention or from disclosed subject matter. The disclosure comprises subject matter consisting of the features defined in the claims or the exemplary embodiments as well as subject matter comprising said features.

Furthermore, in the claims the word “comprising” does not exclude other elements or steps, and the indefinite article “a” or “an” does not exclude a plurality. A single unit or step may fulfil the functions of several features recited in the claims. The mere fact that certain measures are recited in mutually different dependent claims does not indicate that a combination of these measures cannot be used to advantage. The terms “essentially”, “about”, “approximately” and the like in connection with an attribute or a value particularly also define exactly the attribute or exactly the value, respectively. The term “about” in the context of a given numerate value or range refers to a value or range that is, e.g., within 20%, within 10%, within 5%, or within 2% of the given value or range. Components described as coupled or connected may be electrically or mechanically directly coupled, or they may be indirectly coupled via one or more intermediate components. Any reference signs in the claims should not be construed as limiting the scope.

A computer program may be stored/distributed on a suitable medium, such as an optical storage medium or a solid-state medium supplied together with or as part of other hardware, but may also be distributed in other forms, such as via the Internet or other wired or wireless telecommunication systems. In particular, e.g., a computer program can be a computer program product stored on a computer readable medium which computer program product can have computer executable program code adapted to be executed to implement a specific method such as the method according to the invention. Furthermore, a computer program can also be a data structure product or a signal for embodying a specific method such as the method according to the invention.

1. A system for reducing hypersensitivity of a subject, comprising:
a storage device configured to store a plurality of stimuli;
a control circuit coupled to the storage device and configured to select a stimulus of the plurality of stimuli and cause the selected stimulus to be output to the subject;
wherein
the storage device is configured to store difficulty levels for the plurality of stimuli;
the storage device is configured to store a record comprising difficulty levels of previously output stimuli of the plurality of stimuli and time periods of exposure to the previously output stimuli;
the control circuit is configured to use
the stored difficulty levels of the plurality of stimuli,
the difficulty levels of the previously output stimuli stored in the record, and
the time periods of exposure stored in the record for the previously output stimuli
to select the stimulus such that sensory filters of the subject are trained by gradually exposing them to more difficult stimuli, as quantified by the difficulty levels, the selected stimulus comprising a virtual reality, VR, scene, wherein the system enables the subject to navigate through the VR scene, wherein the VR scene comprises a 360° real-world scene.

2. The system of claim 1, further comprising an interface configured to receive a user input from the subject that triggers the control circuit to select the stimulus.

3. The system of claim 2, wherein the user input specifies a desired difficulty level or a desired stimulus, wherein the control circuit is configured to use the user input to select the stimulus.

4. The system of claim 1, wherein the control circuit is configured to store the difficulty level of the selected stimulus in the record when causing the selected stimulus to be output.

5. The system of claim 1, wherein the control circuit is configured to update the record with a time period for which the stimulus was output or with an indicator indicating that a maximum exposure time was reached, for use in selection of a subsequent stimulus.

6. The system of claim 1, wherein the control circuit is configured to process a physiological response of the subject to the selected stimulus to monitor a therapeutic progress.

7. The system of claim 1, further comprising a physiological response measuring device configured to measure the physiological response.

8. The system of claim 1, further comprising a user equipment, UE, of a cellular network configured to communicate with the physiological response measuring device and the control circuit to provide the measured physiological response to the control circuit.

9. The system of claim 1, wherein the physiological response comprises one, several, or all of: a heart rate, a blood pressure, a skin temperature, a skin moisture, a skin conductance, an electrodermal activity, an eye pupil dilation.

10. The system of claim 1, wherein the control circuit is configured to at least one of:
cause the determined therapeutic progress to be output;
cause outputting of the selected stimulus to be terminated in response to detecting from the physiological response that the stimulus causes an unacceptable stress level for the subject.

11. The system of claim 1, further comprising a communication interface configured for secure transmission of data related to the outputting of the selected stimulus, optionally wherein the data comprises the difficulty level of the selected stimulus and an interval length for which the subject was exposed to the selected stimulus.

12. The system of claim 1, wherein the system is configured to output the stimulus based on a performance history specified by the difficulty levels of the previously output stimuli stored in the record and the time periods of exposure stored in the record for the previously output stimuli, so as to cause a gradual increase with respect to both difficulty level and exposure time.

13. The system of claim 1, wherein the system comprises
a computer or a game console, and
VR goggles comprising an optical output device and an acoustic output device,
wherein the computer or game console comprises the control circuit and the storage device and the control circuit is configured to be communicatively coupled to the VR goggles to cause the selected stimulus to be output via the optical output device and/or the acoustic output device.

14. The system of claim 1, wherein the system is a system for treating hypersensitivity of a subject with autism spectrum disorder, attention deficit hyperactivity disorder, traumatic brain injury, stroke, fibromyalgia, post-traumatic stress disorder, complex post-traumatic stress disorder, developmental trauma disorder, other mental disorders.

15. A method of controlling a device that outputs a stimulus to a subject, the method being performed by a control circuit of a system and comprising:
accessing, by the control circuit, a storage device that stores a plurality of stimuli and difficulty levels for the plurality of stimuli;
selecting, by the control circuit, a stimulus of the plurality of stimuli; and
outputting, by the control circuit, control signals or control data to the device that cause the device to output the selected stimulus to the subject;
wherein the storage device stores a record comprising difficulty levels of previously output stimuli of the plurality of stimuli and time periods of exposure to the previously output stimuli,
wherein the control circuit uses
the stored difficulty levels of the plurality of stimuli,
the difficulty levels of the previously output stimuli stored in the record, and
the time periods of exposure stored in the record for the previously output stimuli
to select the stimulus such that sensory filters of the subject are trained by gradually exposing them to more difficult stimuli, as quantified by the difficulty levels, the selected stimulus comprising a virtual reality, VR, scene, wherein the system enables the subject to navigate through the VR scene, wherein the VR scene comprises a 360° real-world scene.

16. The method of claim 15, further comprising updating the record with a time period for which the stimulus was output or with an indicator indicating that a maximum exposure time was reached, for selection of a subsequent stimulus.

17. A machine-readable instruction code which, when executed by one or several programmable circuits, configures the one or several programmable circuits to perform the method of claim 15.
